(19) United States
(12) Patent Application Publication
(10) Pub. No.: US 2022/0296435 A1
(43) Pub. Date: Sep. 22, 2022

(54) Novel Absorbent Wound Dressing Construct That Allows Wound Fluid Assessment In Use

(71) Applicants: KCI Licensing, Inc., San Antonio, TX (US); SYSTAGENIX WOUND MANAGEMENT, LIMITED, West Sussex (GB)

(72) Inventors: Alexander WAITE, North Yorkshire (GB); Thomas Alan EDWARDS, Hampshire (GB); Justin Alexander LONG, Lago Vista, TX (US); Timothy Mark ROBINSON, Shillingstone (GB)

(21) Appl. No.: 17/617,272

(22) PCT Filed: Jun. 4, 2020
(86) PCT No.: PCT/US2020/036034

§ 371 Date: Dec. 7, 2021

Related U.S. Application Data
(60) Provisional application No. 62/858,592, filed on Jun. 7, 2019.

(51) Int. Cl. A61F 13/42; A61F 13/15; A61F 13/505; A61F 13/514; A61F 13/535

(52) U.S. Cl. CPC A61F 13/42; A61F 13/15699; A61F 13/505; A61F 13/514; A61F 13/535; A61F 2013/00846

(57) ABSTRACT

A wound dressing includes a hydrophilic foam layer, a plurality of wicking strips, and a drape layer. The hydrophilic foam layer is configured to engage a wound bed and has a first side and a second side, the second side configured to face the wound bed. The plurality of wicking strips also have a first side and a second side, the second side configured to face the first side of the hydrophilic foam layer. The drape layer also has a first side and a second side, the second side configured to face the first side of the plurality of wicking strips. One or more of the plurality of wicking strips also have a removable tab, wherein the tab extends beyond a periphery of the wound dressing.

### CROSS-REFERENCE TO RELATED APPLICATIONS

This application claims the benefit of priority to U.S. Provisional Application No. 62/858,592, filed on Jun. 7, 2019, which is incorporated herein by reference in its entirety.

### BACKGROUND

The present disclosure relates generally to a wound dressing. The present disclosure relates more particularly to a wound dressing having a hydrophilic foam elastic foam layer, a plurality of wicking strips with removable tabs, and a drape layer, wherein the wicking strips provide an indication of the fluid saturation state of the wound dressing.

It is often desirable to remove wound exudate from a wound bed to promote the healing of the wound. In some cases, wound dressing include a layer of foam configured to absorb the wound exudate from the wound bed. However, there is often no consistent way to indicate when the wound dressing has reached its fluid absorption capacity, so often times a caregiver may not replace the wound dressing as often as desired, or the caregiver may replace the wound dressing too frequently which may retard the healing process, cause additional damage to the wound bed, or cause pain to the patient.

Additionally, it may be desirable to monitor wound exudate in the wound dressing, as it may provide insight as to the condition of the wound. Wound exudate may be sanguinous (red and watery), which could indicate possible blood vessel trauma, or purulent (yellow/green and thick), which could indicate potential infection of the wound. There is often no way to view wound exudate without removing the entire wound dressing before it is necessary, which may also lead to a caregiver replacing the wound dressing too frequently. It is therefore desirable to provide a wound dressing that provides a visual indication of the condition of the absorbed wound exudate as well as a mechanism to sample and perform analysis on the absorbed wound exudate.

### SUMMARY

One implementation of the present disclosure is a wound dressing including a hydrophilic foam layer, a plurality of wicking strips, and a drape layer. The hydrophilic foam layer is configured to engage a wound bed, and has a first side and a second side, the second side configured to face the wound bed. The plurality of wicking strips also has a first side and a second side, the second side configured to face the first side of the hydrophilic foam layer. The drape layer also has a first side and a second side, the second side configured to face the first side of the plurality of wicking strips. One or more of the plurality of wicking strips include a removable tab on a first end, wherein the tab extends beyond a periphery of the wound dressing.

Those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

### BRIEF DESCRIPTION OF THE DRAWINGS

FIG. 1 is a top view of a wound dressing, according to an exemplary embodiment.

FIG. 2 is a bottom view of the wound dressing of FIG. 1, according to an exemplary embodiment.

FIG. 3 is an exploded view illustrating several layers of the wound dressing of FIGS. 1 and 2, according to an exemplary embodiment.

FIG. 4 is an exploded view illustrating several layers of the wound dressing of FIGS. 1 and 2, according to another exemplary embodiment.

FIG. 5 is an exploded view illustrating several layers of the wound dressing of FIGS. 1 and 2, according to another exemplary embodiment.

### DETAILED DESCRIPTION

### Overview

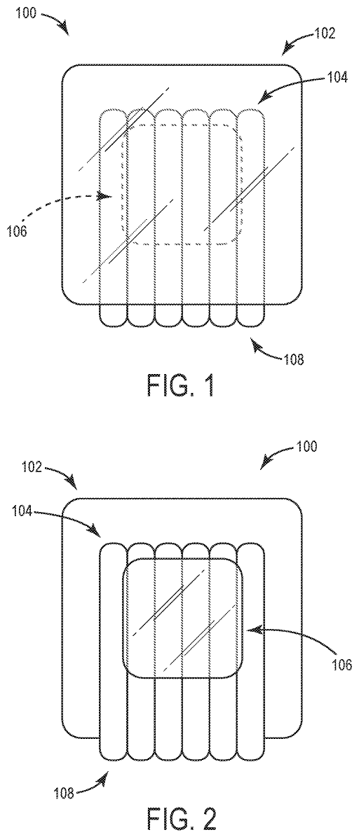

Referring generally to FIGS. 1-5, a wound dressing is shown, according to exemplary embodiments. The wound dressing of the exemplary embodiments has multiple layers including a hydrophilic foam layer and a drape layer. The wound dressing also has a plurality of wicking strips with removable absorbent tabs configured to provide an indication of the fluid absorption within the wound dressing. In some embodiments, the wicking strips may be adjacent to one another, such that they are side-by-side in a horizontal manner. In other embodiments, the wicking strips may be stacked on top of each other, in a vertical manner. In some embodiments, the drape layer and hydrophilic foam layer may be adhered together, such that they surround and encapsulate the wicking strips. In some embodiments, the wound dressing may also have a superabsorbent layer.

Advantageously, the wicking strips of the wound dressing may provide a visual indication of the wound exudate. In some embodiments, the coloration of the hydrophilic foam and the drape layer allows for visual assessment of wound exudate both absorbed in the wound dressing and at the wound site. In some embodiments, the wound dressing is configured to wick wound exudate to the removable absorbent tabs of the wicking strips, which extend beyond the periphery of the wound dressing. A user may remove one of the tabs when it is desirable to perform a further assessment of the wound exudate. The wicking strips may include perforated tear lines to allow the tabs to be easily removed and examined. In some embodiments, the tab can be used to perform a visual assessment of the wound exudate, such as to determine between sanguinous, purulent, healthy, or other conditions of the wound site. The tab may also be further assessed to determine the molecular composition of wound exudate to provide further information in relation to the condition of the wound site and to potentially indicate treatment therapies moving forward. Additionally, this may prevent unnecessary changing of the wound dressing and provide an indication to a caregiver as to when a wound dressing needs to be replaced and the condition of the wound. Additional features and advantages of the wound dressing are described in detail below.

### Wound Dressing

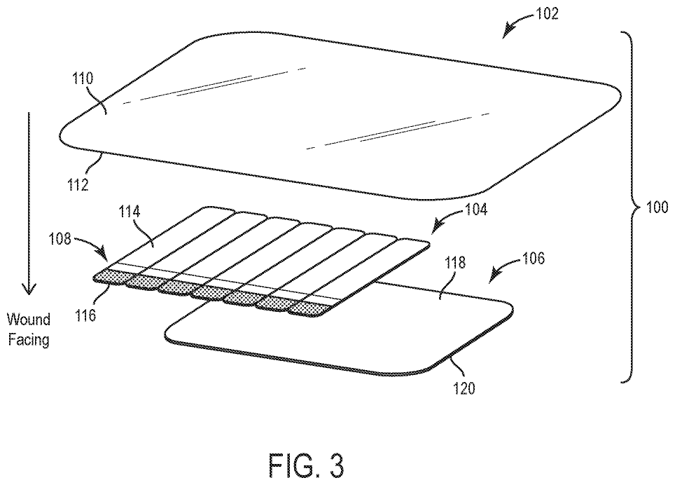

Referring now to FIGS. 1-5, a wound dressing 100 is shown, according to an exemplary embodiment. In a brief overview, FIG. 1 is a top view of a wound dressing 100 as would be visible when wound dressing 100 is adhered to a surface (e.g., a patient's skin). FIG. 2 is a bottom view of wound dressing 100 showing surface of wound dressing 100 configured to contact a wound. FIGS. 3-5 are exploded views of several exemplary embodiments illustrating components and layers 102-108 of wound dressing 100.

In various embodiments, wound dressing 100 can be formed as a substantially flat sheet for topical application to wounds, or formed as a contoured dressing for application to body surfaces having high curvature. The size and shape of wound dressing 100 can vary depending on the size of the wound to be dressed and its location. For example, it is contemplated that the size of wound dressing 100 can range from approximately 1 cm² to 200 cm², and more preferably from approximately 4 cm² to 100 cm². However, other shapes and sizes of wound dressing 100 are also possible depending on the intended use. In other embodiments, wound dressing 100 may have a substantially convex or concave shape, or other customizable topography to adhere to wounds located on areas such as the knee or elbow.

Wound dressing 100 is shown to include (among other possible layers) a drape layer 102, a plurality of wicking strips 104, and a hydrophilic foam layer 106. In some embodiments, the drape layer 102 and hydrophilic foam layer 106 are adhered to each other (e.g., using an adhesive). As shown in FIGS. 1-2, removable tabs 108 of wicking strips 104 may extend beyond a periphery of wound dressing 100, such that they can be accessed by a caregiver. Tabs 108 may allow for a visual assessment and/or analysis of the molecular composition of wound exudate while wound dressing 100 remains in use. Wound dressing 100 may be applied for use at wound bed. In some embodiments, drape layer 102 and hydrophilic foam layer 106 may lie entirely within the confines of a periwound at the wound bed. In other embodiments, the drape layer 102 may extend past a perimeter of hydrophilic foam layer 106 and adhere to a top of the periwound. However, other configurations of the locations of the layers are also possible depending on the intended use, such as the extension of layers into the periwound or the surrounding wound bed.

### Drape Layer

In some embodiments, wound dressing 100 includes a drape layer 102. Drape layer 102 is shown to include a first side 110 and a second side 112 opposite first side 110. Second side 112 is configured to face a wound. When wound dressing 100 is applied to a wound, first side 110 faces away from the wound whereas second side 112 faces towards the wound. Drape layer attaches over hydrophilic foam layer 106. In some embodiments, drape layer 102 is laminated to hydrophilic foam layer 106 using a fusible fiber positioned between drape layer 102 and hydrophilic foam layer 106. Drape layer 102 can be bonded to hydrophilic foam layer 106, for example, by an adhesive or by radiation cross-linking. In some embodiments, drape layer 102 is bonded to the elastic foam layer 106 by urethane or urea linkages. This can be achieved by applying drape layer 102 to hydrophilic foam layer 106 (substantially without mixing) before polyurethane curing is complete. In some embodiments, the adhesive applied to drape layer 102 is moisture vapor transmitting and/or patterned to allow passage of water vapor therethrough. The adhesive may include a continuous moisture vapor transmitting, pressure-sensitive adhesive layer of the type conventionally used for island-type wound dressings (e.g., a polyurethane or polyethylene-based pressure sensitive adhesive). One example of an adhesive which can be used is a pressure sensitive adhesive based on acrylate ester copolymers, polyvinyl ethyl ether and polyurethane, as described in Great Britain Patent Application No. 1280631A. The basis weight of the adhesive may be 20 to 250 g/m², and more preferably 50 to 150 g/m². In some embodiments, after the adhesive has been applied, a surface head (i.e., an iron) is applied to drape layer 102 such that drape layer 102 bonds to the hydrophilic foam layer 106 and wicking strips 104.

In some embodiments, drape layer 102 is a thin layer of polyurethane film. One example of a suitable material for drape layer 102 is the polyurethane film known as ESTANE 5714F. Other suitable polymers for forming drape layer 102 include poly alkoxylalkyl acrylates and methacrylates, such as those described in Great Britain Patent Application No. 1280631A filed Nov. 22, 2002, the entire disclosure of which is incorporated by reference herein. In some embodiments, drape layer 102 includes a continuous layer of a high-density blocked polyurethane foam that is predominantly closed-cell. Drape layer 102 may have a thickness in the range of 10 μm to 100 μm, preferably in the range of 50 μm to 70 μm. In some embodiments, drape layer 102 has a thickness of approximately 60 μm.

Drape layer 102 may be substantially permeable to liquid and moisture vapor. In other words, drape layer 102 may be permeable to both water vapor and liquid water such as wound exudate. Such permeability is intended to facilitate or enhance a hydrophilic gradient from the wound bed, through the wound dressing 100, and to the surrounding atmosphere. In some embodiments, drape layer 102 is impermeable to bacteria and other microorganisms. In other embodiments, drape layer 102 is configured to wick moisture from hydrophilic foam layer 106 and wicking strips 106 to first side 110 of drape layer 102, such that it may evaporate into the atmosphere. In some embodiments, drape layer 102 may be substantially hydrophilic and have a high moisture vapor transmission rate, such as to permit evaporation of would exudate/fluid from first side 110 of drape layer 102.

In the embodiment shown, the perimeter of drape layer 102 extends beyond (e.g., circumscribes) the perimeter of elastic foam layer 106 to provide a margin for wound dressing 100 (e.g., as an “island” dressing) to the skin of a patient adjacent to the wound being treated, and may comprises an adhesive on the second side 112 configured to attach to the wound. Additionally, the adhesive may help attach second side 112 to a first side 114 of wicking strips and a first side 118 of hydrophilic foam layer 106. In other embodiments, the perimeter of drape layer 102 is even with the perimeter of elastic foam layer 106.

In some embodiments, second side 112 of drape layer 102 contacts first side 118 of elastic foam layer 106. Second side 112 of drape layer 102 may simply contact first side 118 of elastic foam layer 106 without the use of an adhesive or may be laminated by fusible fiber or another adhesive to first side 112 of elastic foam layer 106. Second side 112 of drape layer 102 may also contact, in various locations, first side 114 of wicking strips 104. Second side 112 of drape layer 102 may simply contact first side 114 of wicking strips 106 without the use of an adhesive or may be laminated by fusible fiber or another adhesive to first side 114 of wicking strips 104.

### Wicking Strips

In some embodiments, wound dressing 100 includes a plurality of wicking strips 104. Wicking strips 104 are shown to include a first side 114 and a second side 116 opposite the first side 114. Second side 116 is configured to face the wound and contact a first side 118 of hydrophilic foam layer 106. When wound dressing 100 is applied to a wound, first side 114 faces away from the wound whereas second side 116 faces toward the wound. In some embodiments, first side 114 of wicking strips 104 contacts second side 112 of drape layer 102. In some embodiments, first side 114 of wicking strips 104 is adhered to second side 112 of drape layer 102. Alternatively, first side 114 of wicking strips 104 may be in contact with second side 112 of drape layer 102 without the use of adhesive.

In some embodiments, wicking strips 104 may be configured to wick wound exudate from hydrophilic foam layer 106 to removable tabs 108 of wicking strips 104 for inspection. Wicking strips 104 may comprise a variety of materials. In some embodiments, wicking strips 104 may comprise a filter paper, such as Whatman Qualative Filter Paper #1, configured to filter wound exudate absorbed by wound dressing 100. In other embodiments, wicking strips 104 may comprise a viscose, polyester, or cellulose material to facilitate transport of wound exudate from hydrophilic foam layer 106 to removable tabs 108. In some embodiments, wicking strips 104 may be more hydrophilic than hydrophilic foam layer 106, such that they assist in drawing wound exudate through the hydrophilic foam layer 106 and onto a first side 114 of wicking strips 104. Wicking strips 104 may extend from a central portion of wound dressing 100, such as near a center of hydrophilic foam layer 106, to the periphery of wound dressing 100. In other embodiments, wicking strips 104 may extend across the entirety of wound dressing 100 and out to the periphery of wound dressing 100.

In the embodiments shown, wicking strips 104 comprise a plurality of removable tabs 108 on one end of wicking strips 104. In some embodiments, removable tabs 108 can be assessed to determine the molecular and cellular make-up of the absorbed wound exudate composition in order to provide determine future point of care diagnostics in order to dictate treatment strategy. Removable tabs 108 are on an end of wicking strips 104 that extends beyond a periphery of wound dressing 100, such that removable tabs 108 are accessible by a caregiver without removing the entire wound dressing 100. Removable tabs 108 may comprise a variety of materials. In some embodiments, removable tabs 108 may be constructed from an absorbent material, such as a polyurethane foam or a super absorbent polymer, acting to increase the absorptivity of removable tabs 108. In still further embodiments, the absorbent material of removable tabs 108 may be encapsulated in a transparent film envelope, such as a polyurethane film, to assist in the formation of a tab. The material of removable tabs 108 may be white or clear in color so that wound exudate absorbed into removable tabs 108 can be easily viewed by a caregiver. In some embodiments, removable tabs may also have a high degree of fluid retention, acting to hold the wound exudate in place for inspection. In some embodiments, the end of wicking strips 104 that attaches to removable tabs 108 may comprise a tear line or a perforated line, allowing the removable tabs 108 to be easily detached from the wicking strips 104 and wound dressing 100 for assessment. In some embodiments, removable tabs 108 may include a visual indicator, such as a reactive component, which will provide additional information regarding the composition of absorbed wound exudate as well as the condition of the wound. In some embodiments, the visual indicator of the removable tabs 108 may be a pH indicator dye.

In some embodiments, such as the embodiment shown in FIG. 3, wicking strips 104 may be positioned in a side-by-side position, such that they extend across wound dressing 100 horizontally. In the embodiment shown, wound dressing 100 may comprise a total of seven wicking strips 104 and seven removable tabs 108, such that each wicking strip 104 comprises a single removable tab 108. In other embodiments, any number of wicking strips 104 and removable tabs 108 is possible. In an alternate embodiment, wicking strips 104 may each comprise a series of removable tabs 108 to allow for assessment of wound exudate at multiple time points. The removable tabs 108 may be aligned so as to allow for the sequential activation of removable tabs 108, such that a first removable tab 108 must be removed to allow for access to a second removable tab 108. In some embodiments, removable tabs 108 may prevent transport of fluid from one adjacent tab to the next in the sequence until removal of the first removable tab 108. In this instance, wicking strips 104 and the absorbent component of removable tabs 108 may be separated by a fluid impermeable material which is removed upon removal of the removable tab 108, allowing for contact between the wicking strip 104 and the absorbent material of removable tab 108. In the embodiment shown in FIG. 3, each of the wicking strips 104 is the same length and extends to the same central portion of wound dressing 100. In other embodiments, other lengths and configurations of wicking strips 104 are possible.

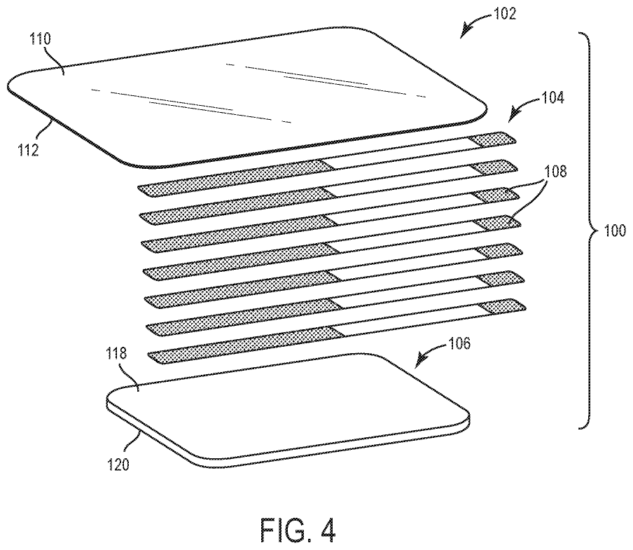

In some embodiments, such as the embodiment shown in FIG. 4, wicking strips 104 may be stacked on top of one another, such that wicking strips 104 are stacked from a second side 112 of drape layer 102 to the first side 118 of hydrophilic foam layer 106. The second side 116 of a first wicking strip 104 may contact the first side 114 of a second wicking strip 104, while the second side 116 of second wicking strip 104 contacts the first side 114 of a third wicking strip 104 and so forth. In some embodiments, a wicking strip 104 in contact with the first side 118 of hydrophilic foam layer 106 may be the first to absorb wound exudate. Wicking strips 104 may comprise a fluid impermeable material/film. Upon removal of the removable tabs 108, the fluid impermeable film may be dislodged, which allows fluid and wound exudate exposure to the next wicking strip 104 in the stack. In the embodiment shown, removable tabs 108 extend past the wound dressing 100 on a single side of wound dressing 100. In other embodiments, wicking strips 104 may be configured to extend in various directions, such that removable tabs 108 fan out from the wound dressing 100 in a plurality of different directions around the perimeter of wound dressing 100.

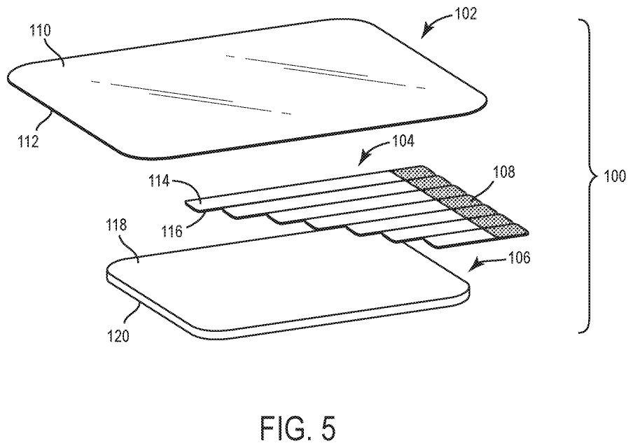

In some embodiments, such as the embodiment shown in FIG. 5, wicking strips 104 may be positioned adjacent to one another, but may be of a variety of lengths. The lengths of wicking strips 104 may coincide with different positions on the hydrophilic foam layer 106 in order to correlate with the saturation level of wound dressing 100. Wicking strips 104 of various lengths may provide an indication of the saturation level of wound dressing 100 as well as the location of dressing saturation. Removable tabs 108 of the various length wicking strips 104 may be examined in order to determine the location and quantity of wound exudate absorbed into wound dressing 10 in order to determine if there is a need for dressing change.

### Hydrophilic Foam Layer

In some embodiments, wound dressing 100 includes a hydrophilic foam layer 106. Hydrophilic foam layer 106 is shown to include a first side 118 and a second side 120 opposite the first side 118. Second side 120 is configured to face and contact the wound. When wound dressing 100 is applied to a wound, first side 118 faces away from the wound whereas second side 120 faces towards the wound. In some embodiments, first side 118 of hydrophilic foam layer 106 contacts second side 112 of drape layer 102 and second side 116 of wicking strips 104. In some embodiments, first side 118 of hydrophilic foam layer 106 may be adhered via an adhesive to second side 112 of drape layer 102 and/or second side 116 of wicking strips 104. Alternatively, first side 118 of hydrophilic foam layer 106 may be in contact with second side 112 of drape layer 102 and/or second side 116 of wicking strips 104 without the use of adhesive.

Hydrophilic foam layer 106 may comprise a polyurethane foam or a polyethylene foam. In some embodiments, hydrophilic foam layer 106 includes a flexible plasticized hydrophilic polymer matrix having an internal cellular structure. Several examples of hydrophilic foams which can be used to make hydrophilic foam layer 106 are described in detail in U.S. Pat. No. 8,097,272 issued Jan. 17, 2012, U.S. Pat. No. 8,664,464 issued Mar. 4, 2014, and U.S. Pat. No. 8,058,499 issued Nov. 15, 2011. The entire disclosure of each of these patents is incorporated by reference herein. In other embodiments, hydrophilic foam layer 106 may be formed from aromatic or aliphatic precursors. Advantageously, hydrophilic foam layer 106 may provide enhanced absorbency for liquid exudate. This is because the porous structure of the foam provides for rapid uptake of liquid exudate.

In some embodiments, hydrophilic foam layer 106 may include a plurality of pores or perforations throughout the hydrophilic foam layer 106. In some embodiments, the perforations may act to allow wound exudate to directly contact wicking strips 104 through hydrophilic foam layer 106. Direct contact of the wound exudate with wicking strips 104 may allow a caregiver to see in real time the condition of wound exudate which may indicate any issues with the wound, as wicking strips 104 facilitate the transfer of wound exudate to the removable tabs 108 on a side of wound dressing 100. Such perforations may also act to filter out undesirable substances from wound fluid exudate. In some embodiments perforations in the hydrophilic foam layer 106 may act to filter out the salt from the wound exudate when the fluid is drawn from the second side 120 to the first side 118 of hydrophilic foam layer 106. In some embodiments, perforations in the hydrophilic foam layer 106 may act to increase the overall flexibility and conformability of wound dressing 100. In some embodiments, the perforations may extend from the first side 118 to the second side 120. In some embodiments, the perforations may be evenly distributed throughout the hydrophilic foam layer 106. In still other embodiments, the perforations may by unevenly distributed through the hydrophilic foam layer 106. In still other embodiments, hydrophilic foam layer 106 may be a solid sheet of material, such that there are no pores or perforations, and wicking strips 104 only contact wound exudate that is passed through hydrophilic foam layer 106.

In some embodiments, wound dressing 100 may also include an absorbent layer (not pictured). An absorbent layer may act to increase the overall absorptivity of wound dressing 100. In some embodiments, the absorbent layer may be a polyurethane foam or a superabsorbent polymer. The superabsorbent polymer may be in the form of granules, including Luquasorb 1160 or 111, such as may be commercially available from BASF, that are contained in a water soluble carrier polymer, such as polyvinylpyrrolidone (PVP). In some embodiments, the absorbent layer may be in the form of a plurality of superabsorbent projections extending from the first side 118 of hydrophilic foam layer 106. In other embodiments, the absorbent layer may be in the form of a continuous sheet of material. In some embodiments, the absorbent layer may be positioned above the hydrophilic foam layer 106, such that first side 118 of hydrophilic foam layer 106 contacts the superabsorbent layer. In other embodiments, the absorbent layer may be positioned between any of the plurality of layers 102-106 of wound dressing 100.

According to an exemplary embodiment, wound dressing 100 has multiple advantages over previous wound dressings. Wound dressing 100 allows for the visual assessment of wound exudate collected in wound dressing 100, which may provide insight into the progression and/or indicate obstacles to healing of the wound. Would dressing 100 also allows for the sampling and extraction of wound exudate during dressing wear, by removable tabs 108 of the wicking strips 104. This provides a caregiver with both a visualization of wound exudate and a method of more closely monitoring the progression of wound healing. Additionally, the reduced need for dressing removal could reduce the overall cost (by reducing the number of wound dressings required) and minimizing patient discomfort and wound disruption caused by the excessing changing of wound dressings

### Configuration of Exemplary Embodiments

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements can be reversed or otherwise varied and the nature or number of discrete elements or positions can be altered or varied. Accordingly, all such modifications are intended to be included within the scope of the present disclosure. The order or sequence of any process or method steps can be varied or re-sequenced according to alternative embodiments. Other substitutions, modifications, changes, and omissions can be made in the design, operating conditions and arrangement of the exemplary embodiments without departing from the scope of the present disclosure.

What is claimed is:

1. A wound dressing, comprising:
a hydrophilic foam layer configured for placement proximate a wound bed, and having a first side and a second side, the second side configured to face the wound bed;
a plurality of wicking strips having a first side and a second side, the second side configured to face the first side of the hydrophilic foam layer; and
a drape layer having a first side and a second side, the second side configured to face the first side of the plurality of wicking strips,
wherein one or more of the plurality of wicking strips comprise a removable tab on a first end, wherein the tab extends beyond a periphery of the wound dressing.

2. The wound dressing of claim 1, wherein the plurality of wicking strips are positioned side-by-side, such that a first wicking strip is adjacent to a second wicking strip.

3. The wound dressing of claim 1, wherein the plurality of wicking strips comprise a plurality of different lengths.

4. The wound dressing of claim 1, wherein the plurality of wicking strips comprise the same length.

5. The wound dressing of claim 1, wherein the plurality of wicking strips are stacked vertically on top of one another.

6. The wound dressing of claim 1, wherein the wicking strips comprise a material configured to wick wound exudate from the hydrophilic foam layer to the removable tabs of the wicking strips.

7. The wound dressing of claim 1, wherein the wicking strips comprise a filter paper.

8. The wound dressing of claim 6, wherein the wicking strips comprise a viscose, polyester, or cellulose material.

9. The wound dressing of claim 1, wherein the wicking strips extend from a central portion of the wound dressing to the periphery of the wound dressing.

10. The wound dressing of claim 1, wherein the wicking strips extend to the periphery of the wound dressing on a single side of the wound dressing.

11. The wound dressing of claim 1, wherein the wicking strips extend to the periphery of the wound dressing on a plurality of sides of the wound dressing.

12. The wound dressing of claim 1, wherein the removable tabs are configured to provide a visual indication as to the condition of absorbed wound exudate.

13. The wound dressing of claim 1, wherein the removable tabs comprise an absorbent material.

14. The wound dressing of claim 13, wherein the removable tabs comprise at least one of a polyurethane foam or a super absorbent polymer.

15. The wound dressing of claim 13, wherein the absorbent material of the removable tabs is encapsulated in a transparent film.

16. The wound dressing of claim 1, wherein the removable tabs comprise a reactive component configured to provide information to a caregiver regarding composition of the wound exudate.

17. The wound dressing of claim 16, wherein the reactive component is a pH indicator dye.

18. The wound dressing of claim 1, wherein the wicking strips comprise a perforated tear line attaching the removable tabs.

19. The wound dressing of claim 1, wherein the removable tabs are configured to have a high degree of fluid retention to retain absorbed wound exudate.

20. The wound dressing of claim 1, wherein the drape layer comprises a polyurethane film.

21. The wound dressing of claim 1, wherein the drape layer is adhered to one or more of the hydrophilic foam layer and the plurality of wicking strips.

22. The wound dressing of claim 1, wherein the hydrophilic foam layer comprises one of a polyurethane foam or a polyethylene foam.

23. The wound dressing of claim 1, further comprising an absorbent layer.

24. A method of making a wound dressing, comprising:
providing a hydrophilic foam layer having a first side and a second side, the second side configured to face a wound bed;
assembling a plurality of wicking strips to the hydrophilic foam layer, the wicking strips having a removable tab extending at least partially beyond the hydrophilic foam layer; and
covering the hydrophilic foam layer and the wicking strips with a drape layer, so that the removable tabs extend at least partially beyond a periphery of the drape layer.

25. The method of claim 24, wherein the plurality of wicking strips are positioned side-by-side, such that a first wicking strip is adjacent to a second wicking strip.

26. The method of claim 24, wherein the plurality of wicking strips comprise a plurality of different lengths.

27. The method of claim 24, wherein the plurality of wicking strips are substantially the same length.

28. The method of claim 24, wherein the plurality of wicking strips are stacked vertically on top of one another.
